(19) United States
(12) Patent Application Publication
(10) Pub. No.: US 2025/0218536 A1
(43) Pub. Date: Jul. 3, 2025

(54) IDENTIFICATION, CHARACTERIZATION, AND QUANTITATION OF CRISPR-INTRODUCED DOUBLE-STRANDED DNA BREAK REPAIRS

(71) Applicant: INTEGRATED DNA TECHNOLOGIES, INC., Coralville, IA (US)

(72) Inventors: Heng Li, Wellesley, MA (US); Gavin Kurgan, Iowa City, IA (US); Matthew McNeill, Iowa City, IA (US); Yu Wang, North Grafton, MA (US)

(21) Appl. No.: 19/052,059

(22) Filed: Feb. 12, 2025

Related U.S. Application Data
(63) Continuation of application No. 16/919,577, filed on Jul. 2, 2020, now Pat. No. 12,254,959.
(60) Provisional application No. 62/870,426, filed on Jul. 3, 2019.
(60) Provisional application No. 62/870,471, filed on Jul. 3, 2019.
(60) Provisional application No. 62/952,603, filed on Dec. 23, 2019.
(60) Provisional application No. 62/952,598, filed on Dec. 23, 2019.

(51) Int. Cl. G16B 20/30; G16B 20/20; G16B 30/10; G16B 40/20

(52) U.S. Cl. CPC G16B 20/30; G16B 20/20; G16B 30/10; G16B 40/20

(57) ABSTRACT

Described herein is a system and process for identifying and characterizing double-stranded DNA break repair sites that are based on biological information and have improved accuracy. Also described is a sequence alignment process that uses biological data to inform the alignment matrix for position specific alignment scoring, resulting in the identification of noncanonical target sites.

### CROSS-REFERENCE TO RELATED APPLICATIONS

This application is a continuation of U.S. patent application Ser. No. 16/919,577, filed on Jul. 2, 2020, which claims priority to U.S. Provisional Patent Application Nos. 62/870,426 and 62/870,471, both filed on Jul. 3, 2019, and 62/952,603 and 62/952,598, both filed on Dec. 23, 2019, each of which are incorporated by reference herein in its entirety.

### TECHNICAL FIELD

Described herein is a system and process for identifying and characterizing double-stranded DNA break repair sites that are based on biological information and have improved accuracy. Also described is a sequence alignment process that uses biological data to inform the alignment matrix for position specific alignment scoring, resulting in the accurate identification of noncanonical target sites.

### BACKGROUND

The use of targeted nucleases, such as CRISPR proteins, has transformed genome editing. CRISPR enzymes form ribonucleoproteins (RNPs) when hybridized with either 2-part crRNA and tracrRNA or single guide RNAs (sgRNAs). With either approach, a short protospacer sequence (a guide RNA or “gRNA”) targets a specific sequence in a complementary molecule. Upon finding a match, these enzymes introduce a break in one or both DNA (or RNA) strands. CRISPR enzymes targeting DNA (e.g., Cas9, Cas12a/Cpf1) introduce double stranded breaks (DSBs) at predictable genomic positions relative to the gRNA's hybridization target. DNA DSBs are repaired by intracellular machinery, but the repair process often results in insertions and deletions (indels), substitutions, and other suboptimal allelic variants.

Because each cell in an affected population must repair itself independent of adjacent cells, and the specific outcome can contain different resulting alleles, the population of cells is likely to contain a plurality of alleles at the targeted location. Additionally, the targeting capability of these nucleases is often somewhat non-specific, which results in undesired mutations at other, off-target, genomic locations.

It is highly desirable to characterize and quantify the plurality of alleles at both on-target and off-target locations. Researchers often use DNA sequencing (such as Illumina next generation sequencing; NGS) to observe the resulting allelic diversity. Multiplexed polymerase chain reaction (PCR) can be performed to amplify and enrich all targeted locations. The resulting amplicons can be sequenced. The plurality of alleles can be characterized and counted using specialized software.

Many specialized software tools have been developed to characterize the allelic variants resulting from DSBs. Prior tools include CRISPResso [1], crispRvariants [2], and Amplican [3]. These tools generally work by aligning each sequence read against expected amplicon targets using the Needleman-Wunsch, bwa, or custom alignment algorithms. The algorithm generates a list of possible read: target alignments. Each alignment is scored based on the number of matching, mismatching, and missing nucleotides (gaps). The best scoring alignment is used for downstream data processing.

Alignment algorithms sometimes generate equally valued query: target alignments, which are most likely to occur when the query contains an insertion or deletion. From the equally valued options, alignment methods will return all or select one option. When selecting, some methods make the selection at random. Without a good predictive model or set of heuristic rules to make the selection, the alignment choice is variable, which can lead to incorrect indel annotations and lower accuracy results.

What is needed is an algorithm and process for identifying and characterizing double-stranded DNA break repair sites that are based on biological information and have improved accuracy.

### SUMMARY

One embodiment described herein is a computer implemented process for identifying and characterizing double-stranded DNA break repair sites with improved accuracy, the process comprising executing on a processor the steps of: receiving sample sequence data comprising a plurality of sequences; analyzing and merging of the sample sequence data and outputting merged sequences; developing target-site sequences containing predicted outcomes of repair events when a single-stranded or a double-stranded DNA oligonucleotide donor is provided and outputting the target predicted outcomes; binning the merged sequences with the target-site sequences or the optional target predicted outcomes using a mapper and outputting target-read alignments; re-aligning the binned target-read alignments to the target-site using an enzyme specific position-specific scoring matrix derived from biological data that is applied based on the position of a guide sequence and a canonical enzyme-specific cut site and producing a final alignment; analyzing the final alignment and identifying and quantifying mutations within a pre-defined sequence distance window from the canonical enzyme-specific cut sites; outputting the final alignment, analysis, and quantification results data as tables or graphics. In one aspect, the sequence data comprises sequences from a population of cells or subjects. In another aspect, the enzyme-specific cut site comprises one or more of Cas9, Cas12a, or other Cas enzymes. In another aspect, the pre-defined sequence distance window is enzyme specific and comprises between 1 nt to about 15 nt. In another aspect, the results show the percent editing, percent insertion, percent deletion, or a combination thereof. In another aspect, the accuracy of identifying variant target sites is improved by about 15 to about 20% as compared to comparable processes.

Another embodiment described herein is a computer implemented process for aligning biological sequences, the process comprising executing on a processor the steps of: receiving sample sequence data comprising a plurality of sequences; aligning the sequence data to a predicted target sequence using a matrix based on an enzyme specific position-specific scoring of a specific nuclease target site; outputting the alignment results as tables or graphics. In one aspect, the sequence data comprises sequences from a population of cells or subjects. In another aspect, the specific nuclease target sequence comprises a target site for one or more of Cas9, Cas12a, or other Cas enzymes. In another aspect, the matrix uses position-specific gap open and extension penalties.

Another embodiment described herein is a method for identifying and characterizing double-stranded DNA break repair sites with improved accuracy, the process comprising: extracting genomic DNA from a population of cells or tissue from a subject; amplifying the genomic DNA using multiplex PCR to produce amplicons enriched for target-site sequences; sequencing the amplicons and obtaining sample sequence data; subsequently executing on a processor, the steps of: receiving sample sequence data comprising a plurality of sequences; analyzing and merging of the sample sequence data and outputting merged sequences; developing target-site sequences containing predicted outcomes of repair events when a single-stranded or a double-stranded DNA oligonucleotide donor is provided and outputting the target predicted outcomes; binning the merged sequences with the target-site sequences or the optional target predicted outcomes using a mapper and outputting target-read alignments; re-aligning the binned target-read alignments to the target-site using an enzyme specific position-specific scoring matrix derived from biological data that is applied based on the position of a guide sequence and a canonical enzyme-specific cut site and producing a final alignment; analyzing the final alignment and identifying and quantifying mutations within a pre-defined sequence distance window from the canonical enzyme-specific cut sites; outputting the final alignment, analysis, and quantification results data as tables or graphics. In one aspect, the enzyme-specific cut site comprises one or more of Cas9, Cas12a, or other Cas enzymes. In another aspect, the pre-defined sequence distance window is enzyme specific and comprises between 1 nt to about 15 nt. In another aspect, the results show the percent editing, percent insertion, percent deletion, or a combination thereof. In another aspect, the accuracy of identifying variant target sites is improved by about 15 to about 20% as compared to comparable processes.

### DESCRIPTION OF THE DRAWINGS

FIG. 1 shows a general workflow for CRISPAltRations. Edited genomic DNA is extracted and amplified using targeted multiplex PCR to enrich for the on- and predicted off-target loci. Amplicons are sequenced on an Illumina MiSeq. Read pairs are merged into a single fragment (FLASH), mapped to the genome (minimap2) and binned by their alignment to expected amplicon positions. Reads in each bin are re-aligned to the expected amplicon sequence after finding the cut site and creating a position specific gap open/extension bonus matrix to preferentially align indels closer to the cut site/expected indel profiles for each enzyme (CRISPAItRations code+psnw). Indels that intersected with a window upstream or downstream of the cut site were annotated. Percent editing is the sum of reads containing indels/total observed.

FIG. 2 shows a directed acyclic graphic of the CRISPRAltRations pipeline. Dashed boxes represent steps within the pipeline, and each step may contain one or more software tools. Lines and arrows show the flow of information through the pipeline. Two important steps are minimap2_orig_reads (orange) uses minimap2 [4] to align sequence reads against a reference genome, which is an optional step. Later, the minimap2 is used to align sequence reads against their expected target regions. The CRISPy Python tool (developed internally), re-aligns sequence reads against their target regions by calling a custom-modified version of psnw, which performs a cut-site re-alignment against the target region. CRISPy is also responsible for characterizing the detected indels in aligned reads.

FIG. 3 shows MinION sequencing read data aligned to a reference target demonstrates a blunt insertion. The drop in expected coverage (grey highlight) indicates a large insertion. The mismatches on the internal edges of the grey highlight indicate mismatches between the observed read data and the expected reference.

FIG. 4A-B show cartoon examples representing the kinds of structural variants that could occur when using a DNA oligo as a template for homology directed repair (HDR). These examples are not comprehensive. In the cartoon, blue represents the reference sequence, green represents the homology arms, and orange represents the desired inserted sequence. FIG. 4A-B show exemplary double stranded and single stranded template oligos, respectively. (1) [Prefect repair]—the region containing the DSB is repaired without any structural variants being introduced even when single or double stranded template oligos are present. (2) [HDR-mediated repair]—the template oligo directs HDR, resulting in the desired insertion. Only the desired inserted sequence is observed in the repaired DNA. (3) [Non-homologous end joining (NHEJ) repair]—the template oligo is inserted bluntly after the DSB. (4) [NHEJ repair with duplicate insertion]—the template oligo is inserted bluntly multiple times after the DSB. Examples 3 and 4 are most likely to occur with a double stranded oligo used as a repair template; the homology arms present in the donor sequence are also inserted into genome.

FIG. 5A-B show position of occurrence of editing events in Cas9 data. Position of indel occurrence from the nuclease cut site using a set of 274 Cas9 guides editing unique genomic targets in a Jurkat cell line. Both deletion (FIG. 5A) and insertion (FIG. 5B) position events were normalized by quantifying them as % total of the respective indel event within the sample. Only sites with >50 reads and >5% indels were used for analysis to limit low-confidence signal and remove noise. Indels were quantified within a 20 bp window from the cut site. Outliers were largely found to be sites that had a non-reference indel present in the Jurkat cell line that did not appear to be caused by DSB activity.

FIG. 6A-B show position of occurrence of editing events in Cas12a data. Position of indel occurrence from the nuclease cut site using a set of 199 Cas12a guides editing unique genomic targets in a Jurkat cell line. Both deletion (FIG. 6A) and insertion (FIG. 6B) position events were normalized by quantifying them as % total of the respective indel event within the sample. Only sites with >50 reads and >5% indels were used for analysis to limit low-confidence signal noise. Indels were quantified within a 20-bp window from the cut site. Outliers were largely found to be sites that had a non-reference indel present in the Jurkat cell line that did not appear to be caused by DSB activity.

FIG. 7A-B show gap open/extension penalty vectors near the nuclease introduced DSB for Cas9 (FIG. 7A) or Cas 12a (FIG. 7B). To positively weight indels near the cut site in sequence alignments, we use a position specific matrix of values representing variable gap open or extension penalties are used, where the length of the array is equal to the integer positions of each nucleic acid in the target (thick blue lines). The vector values (red circles and blue diamonds) are varied based on the proximity to the nuclease cut site (vertical black dashed line). Thus, indels that are closest to the cut site have the least gap open or extension penalty.

FIG. 8A-C show choosing an optimal window to observe true CRISPR-Cas9 of Cas12 editing events. FIG. 8A-B shows the editing window is a nucleotide distance around the CRISPR cut site used to identify indels. True and false editing events were calculated as % indels from targeted sequencing of cells treated (FIG. 8A) or untreated (FIG. 8B) for Cas9 (n=263 pairs of treatment vs. control) or (FIG. 8C) Cas12a (n=384 pairs of treatment vs. control), respectively. Most true editing events are collected with a 4 nt window for Cas9 or 7 nt for Cas12a; while expanding the window only further collects additional false editing.

FIG. 9 shows a screen shot of deduplicated read alignments by observed frequency. Total region coverage is indicated by the height of the vertical gray bars (top), and reads are horizontal colored bars. Brighter colored reads indicate more frequently observed indels. Horizontal thin lines indicate deletions; vertical purple “|” symbols show insertions, and colored bars within reads indicate mismatched bases.

FIG. 10 shows that CRISPAltRations reliably finds the correct indel. Each bar reports the percentage of correctly returned indels. Using synthetically generated data representing 12,060 unique indel events, evenly distributed between each size bin (x-axis) and 603 unique amplicons. Error bars represent the 95% confidence interval across targets.

FIG. 11A-B show that CRISPRAltRations more accurately reports the total % editing at the 603 synthetic targets with simulated Cas9 (FIG. 11A) or Cas12a (FIG. 11B) editing. Dots represent the percent editing observed at each target site, and the horizontal line represents the percent indels introduced at each target in the generated synthetic data. CRISPResso2Pooled and CRISPResso1Pooled were run using default pipeline parameters for each panel with the required minimal read depth for quantification removed (to prevent incorrect drop-out of amplicons).

### DETAILED DESCRIPTION

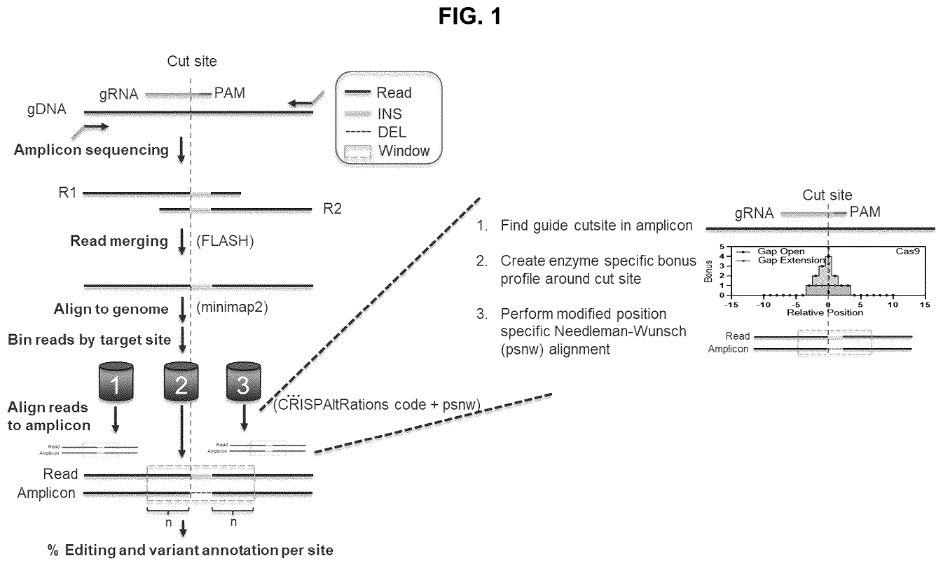

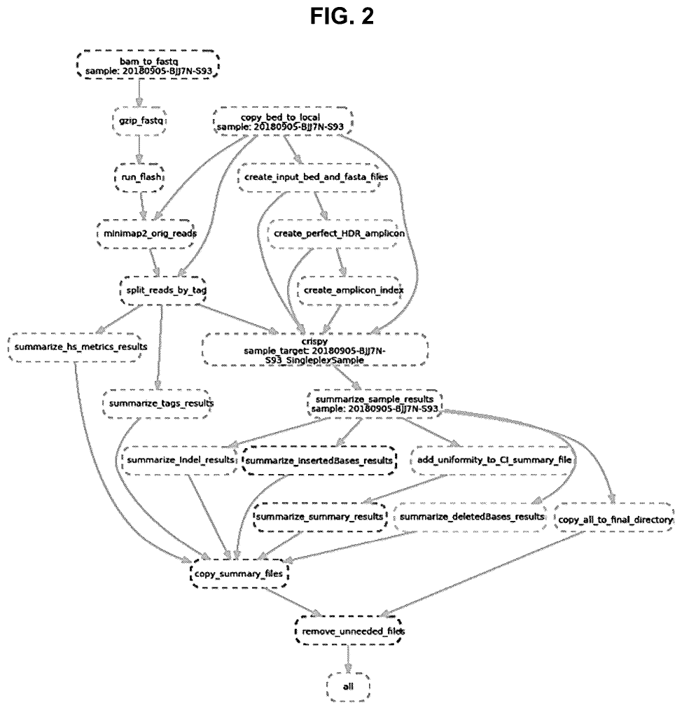

One embodiment described herein is an analytical pipeline called CRISPAltRations. See FIGS. 1-2. Briefly, this pipeline takes in FASTQ files, and builds a merged R₁/R₂ consensus using FLASH. Simultaneously, a target site reference is built, which describes the sequences for all expected on-target locations. Optionally, a target is built that contains an expected outcome of a homology directed repair (HDR) event. Next, the merged sequence reads are aligned to the target reference sequences using minimap2, (which was originally developed for rapid alignment of long reads (e.g., those generated by the Oxford Nanopore Technologies MinION). Reads aligning to each target are then re-aligned using a modified form of the Smith-Waterman aligner. The modified aligner allows us to improve detection of insertions and deletions resulting from DSB repair. All observed variants within a pre-defined distance of the DSB location are characterized and quantified. Finally, the results are summarized in tables and graphs. The various described programs, tools, and file types (as well as those mentioned below) are familiar to and readily accessible to those having ordinary skill in the art. It should be understood that these programs, tools, and file types are exemplary and are not intended to be limiting. Other tools and file types could be used to practice the described processing and analysis.

In this analytical pipeline, the following improvements over prior methods are described. First, the use of minimap2 [4] enables alignment of reads generated from both short and long read sequencers. Second, by constructing the expected outcome of the homology directed repair event, the ability to characterize perfect (i.e., correctly occurring) HDR events is improved. Third, use of the modified Needleman-Wunsch aligner that can accept a Cas-specific bonus matrix enables significantly improved indel characterization and percent (%) editing quantification over prior methods. Fourth, graphical visualization of the introduced allelic variants is improved. Fifth, a predicted repair event, as described in a prior tool [5], is compared against the observed repair, and the molecular pathways involved in the repair can be described.

In one embodiment, the processes described herein have the following advantageous uses:
Accurate characterization of indel profiles resulting from DSBs.
The fraction of reads containing an indel after a DSB is repaired is used to calculate the percentage of editing. This metric (% editing) is used to determine the effectiveness of a gRNA for use in CRISPR-Cas gene editing.
Accurate characterization of the resulting indel similarly improves the ability to identify the percentage of cellular chromosomes in a population of cells containing a frame-shifting mutation. Frame-shifting mutations modify proteins encoded by affected genes.
Accurate characterization of inserted sequences.
Accurate characterization of multiple mutations resulting from multiple gRNA/Cas9 (i.e., ribonucleoprotein complex) deliveries or dual-guide region modifications.
Analysis of indels sequenced on a long-read platform, such as MinION. Additionally, it allows phased characterization of both ends of large (>400 nt) insertion of deletion events, which occur after DSB repair.
Improved result visualization.

One embodiment described herein is a computer implemented process for identifying and characterizing double-stranded DNA break repair sites with improved accuracy, the process comprising executing on a processor the steps of: receiving sample sequence data comprising a plurality of sequences; analyzing and merging of the sample sequence data and outputting merged sequences; developing target-site sequences containing predicted outcomes of repair events when a single-stranded or a double-stranded DNA oligonucleotide donor is provided and outputting the target predicted outcomes; binning the merged sequences with the target-site sequences or the optional target predicted outcomes using a mapper and outputting target-read alignments; re-aligning the binned target-read alignments to the target-site using an enzyme specific position-specific scoring matrix derived from biological data that is applied based on the position of a guide sequence and a canonical enzyme-specific cut site and producing a final alignment; analyzing the final alignment and identifying and quantifying mutations within a pre-defined sequence distance window from the canonical enzyme-specific cut sites; outputting the final alignment, analysis, and quantification results data as tables or graphics.

In one embodiment, edited genomic DNA is extracted and amplified using targeted multiplex PCR to enrich for the on- and predicted off-target loci. Amplicons are sequenced on an Illumina MiSeq. Read pairs are merged into a single fragment (FLASH), mapped to the genome (minimap2) and binned by their alignment to expected amplicon positions. Reads in each bin are re-aligned to the expected amplicon sequence after finding the cut site and creating a position specific gap open/extension bonus matrix to preferentially align indels closer to the cut site/expected indel profiles for each enzyme (CRISPAltRations code+psnw). Indels that intersected with a window upstream or downstream of the cut site were annotated. Percent editing is the sum of reads containing indels/total observed.

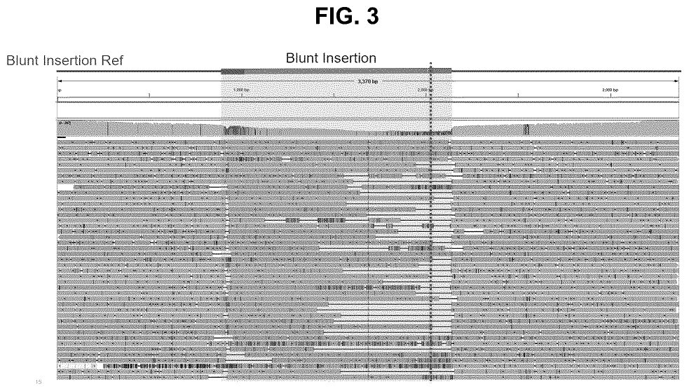

In some embodiments, the process described herein uses minimap2 [4], which enables alignment of reads generated from both short and long read sequencers. Prior tools typically only accept short read sequencing data, such as those that are generated by Illumina sequencers. Others have used long read sequencing data to examine large insertions or deletions [6-8], but no stand-alone publicly available tools are believed to exist. Long read data handling is partially enabled by use of the minimap2 aligner. For example, the alignment results can be visualized, which shows identification of a blunt molecular insertion in DNA after a DSB repair (FIG. 3). Another embodiment sorts between real and noise-derived indels using a method similar to a previously published tool [7] in which small indels are ignored.

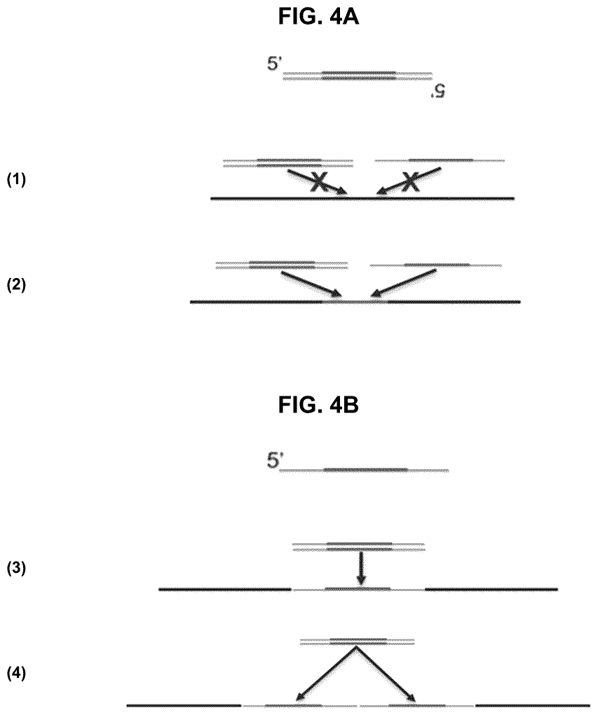

In another embodiment, by constructing the expected outcome of the HDR event, the ability to characterize perfect HDR events is improved. A reference file, in FASTA format, contains each expected sequence target and modified sequence targets as well. The first step toward constructing this file involves creating a reference sequence index that enables reads to be aligned to each expected structural variant. For example, if one interrogates a region targeted for a DSB and double stranded DNA donor oligo to enable HDR, there are multiple different likely biological repair outcomes: perfect repair (FIG. 4-1), HDR-mediated repair (FIG. 4-2), NHEJ repair (FIG. 4-3), and NHEJ repair with duplicate insertion (FIG. 4-4). Other outcomes, such as template fragment or triple template insertions, are also possible (not shown). A similar reference file construction approach has been used by other tools, such as UDiTaS™ [9].

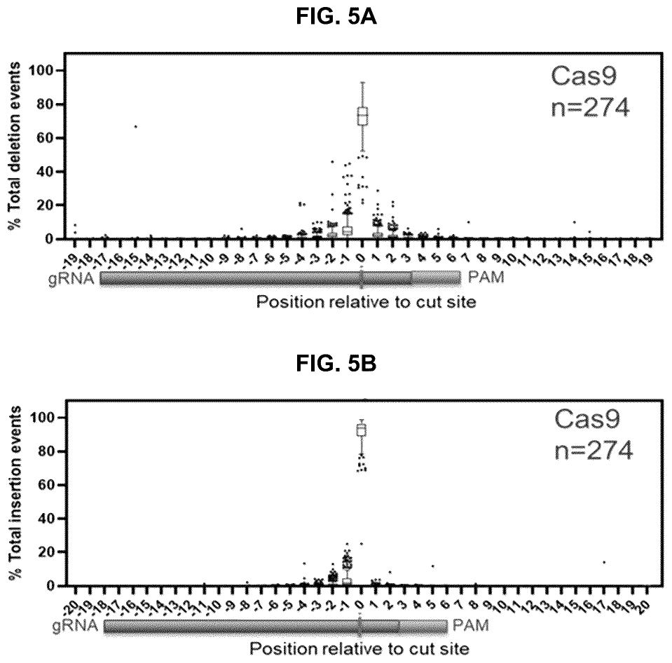

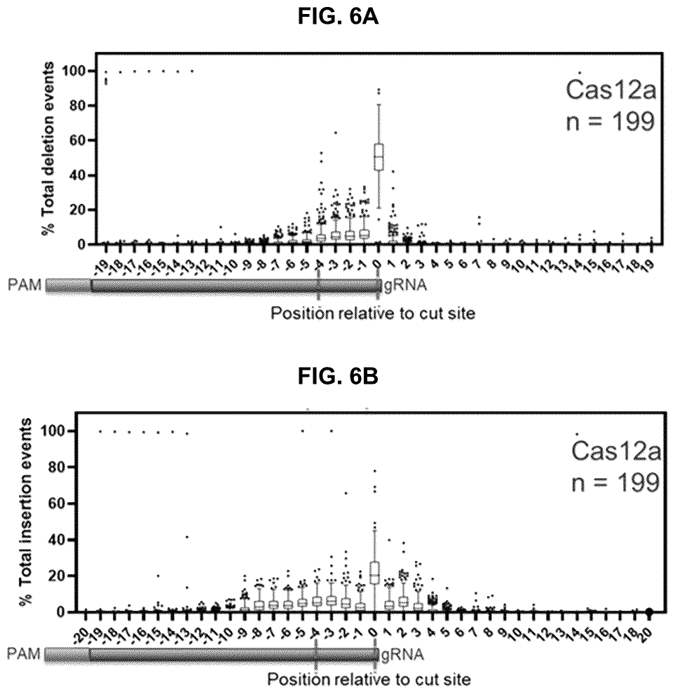

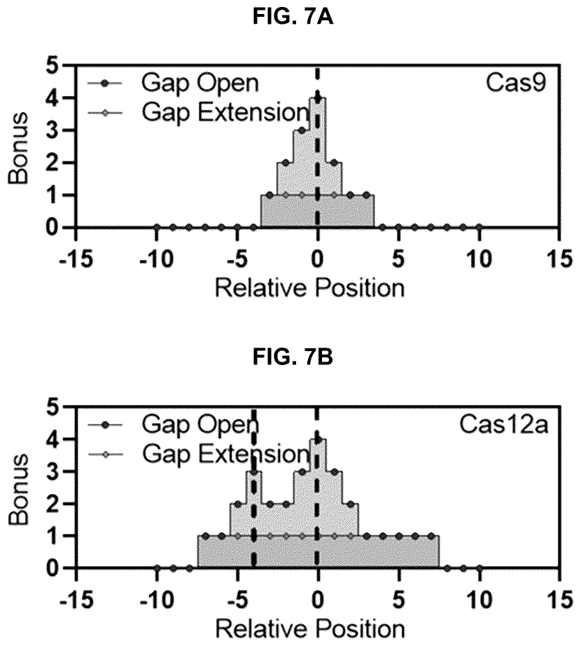

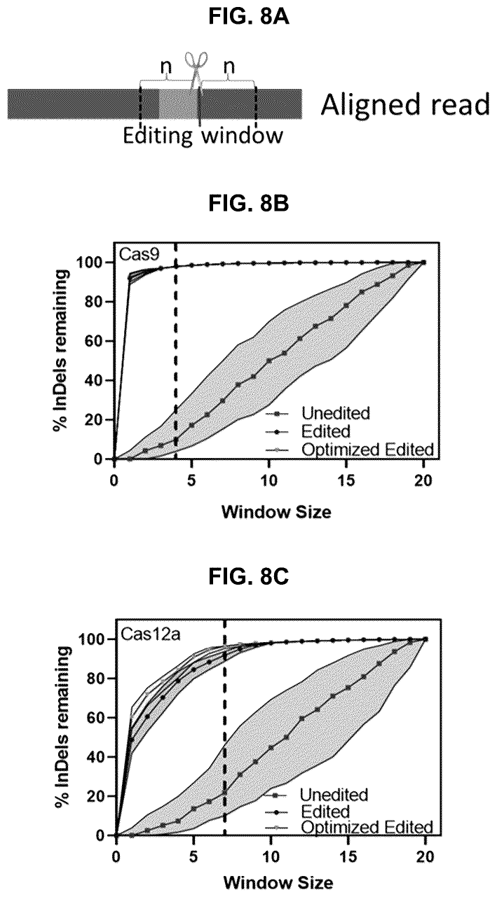

In another embodiment, a modified version of the Needleman-Wunsch algorithm is used to re-align reads against their expected target. The method described herein increases accuracy of alignments containing an indel (as annotated in alignment's CIGAR string). It significantly improves indel characterization and % editing quantification over prior methods. DNA sequence aligners such as minimap2 and Needleman-Wunsch approaches weigh indel alignments using fixed penalties for opening and extending gaps. This method is improved upon by re-aligning reads to their targets using position-specific gap open and extension penalties (enabled in a tool called “psnw”) such that alignments with indels favor positioning them overlapping or near the predicted DSB. This position specific matrix is set to reflect the actual characterized indel profile of the specific Cas enzyme being used for editing (FIGS. 5-6). Thus, indel base alignments are most highly favored at or near the predicted target cut site (variable scoring strategy; FIG. 7). This method enables accurate realignment of indels, particularly those that occur in repetitive regions in the reference sequence. This approach improves the ability to identify the most biologically likely result.

A recently developed tool (CRISPResso2 [11]), uses a cut-site aware alignment strategy. However, the processes described herein implements the cut-site aware alignment method using a full gap open/extension matrix during alignment that is tuned by actual editing data at Cas9/Cas12a sites and implemented in C++. In contrast, CRISPResso2 uses a method that only enables a single bonus at the cut site and is implemented in Python.

In another embodiment, the processes described herein collect indels nearby the nuclease cut site and tag indels that intersect the cut site, or within a fixed distance. Some published accounts suggest a 1-2 nt fixed distance, but the data supporting those choices has been limited. In developing the embodiments described herein, the optimal distance (i.e., window size) around the cut site was studied using a set of Cas9-RNP treated and paired untreated control samples. It was observed that a 4-nt window for Cas9 or a 7-nt window for Cas12a provided the greatest sensitivity and provided an acceptable specificity (FIG. 4). The larger window requirement for Cas12a is likely due to the mechanism of action; Cas12a implements a double strand break by producing two single strand breaks 5-bp away (leaving “sticky” ends) [12]. Thus, the process described herein can be expanded to other nucleases (e.g., CasX) [13] having biological data to inform the target window size and enzymatic mechanism of action.

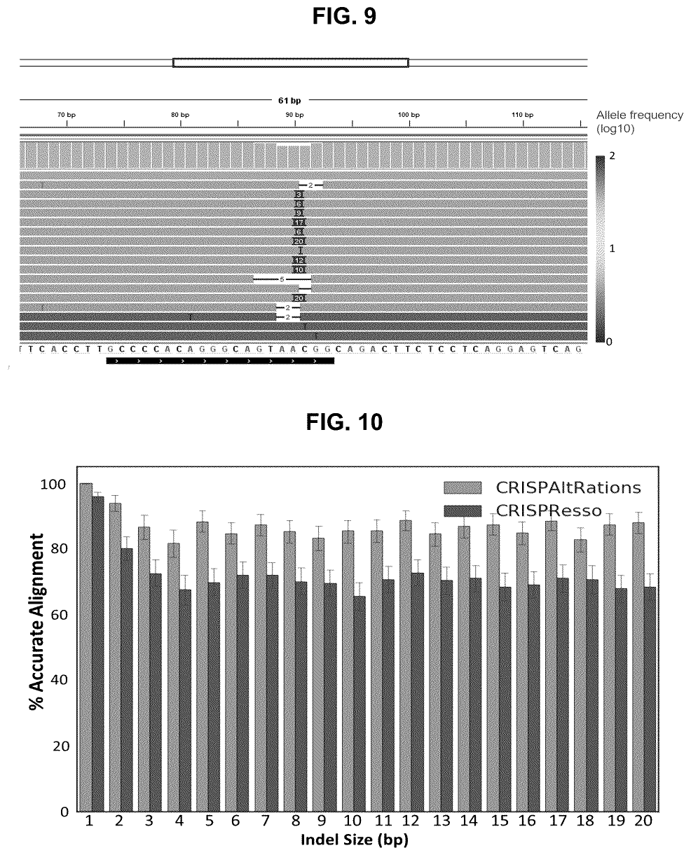

In another embodiment, graphical visualization of the allelic variation is improved. Downstream of the alignment step, several other analyses are performed that are unique to the described method. To generate an improved visualization, reads are deduplicated based on the identity of identified indel sequences within the CRISPR editing window post-alignment. Deduplicated reads are written back to a BAM file, and the frequency of each deduplicated read within the original population of reads is written to an associated BAM tag. After the file is indexed, indels in deduplicated reads and their associated frequencies can be visualized using the commonly available IGV tool [10] (FIG. 9).

In another embodiment, a predicted repair event is compared, as described in a prior tool [5], against the observed repair, and can be used to determine the molecular pathways involved in the repair. The system described herein also adds the ability to compare the observed indel profile against expected indel profiles, which enables rapid discernment about whether an experimental treatment modified the intracellular mechanisms of DNA repair.

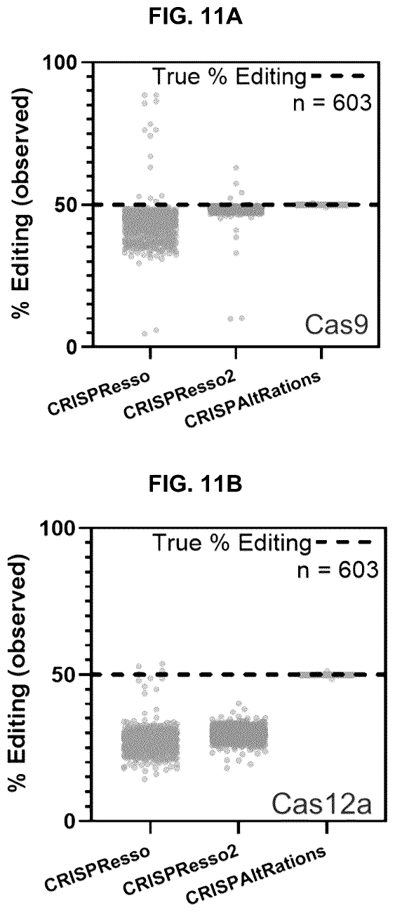

The utility of the system and methods described herein is demonstrated by generating a synthetic set of 603 gRNA: amplicon pairs. At each target, 4000 read pairs (2×150 bp) are synthetically generated with a simulated Illumina MiSeq v3 platform error profile. In half of the reads, random indels are introduced based on a model generated off the observed editing profile for Cas9 and Cas12a (FIGS. 4-5). The synthetic data is analyzed using the CRISPRAltRations system described herein and the previously published CRISPResso1 and CRISPResso2 tools [11]. By implementing the method described herein, the ability to correctly characterize indels is improved by ˜15-20% (FIG. 10). The algorithm described herein has increased accuracy because it produces a biologically informed selection of the best alignment in targets where multiple equally scored alignments are possible. Additionally, the method described herein more accurately calculates the percentage of modified DNA molecules (FIG. 11). The process and strategy described herein is an important enhancement toward characterizing and quantifying indels introduced after DSB repair.

Another embodiment described herein is a computer implemented process for aligning biological sequences, the process comprising executing on a processor the steps of: receiving sample sequence data comprising a plurality of sequences; aligning the sequence data to a predicted target sequence using a matrix based on an enzyme specific position-specific scoring of a specific nuclease target site; outputting the alignment results as tables or graphics. In one aspect, the sequence data comprises sequences from a population of cells or subjects. In another aspect, the specific nuclease target sequence comprises a target site for one or more of Cas9, Cas12a, or other Cas enzymes. In another aspect, the matrix uses position-specific gap open and extension penalties.

Another embodiment described herein is a method for identifying and characterizing double-stranded DNA break repair sites with improved accuracy, the process comprising: extracting genomic DNA from a population of cells or tissue from a subject; amplifying the genomic DNA using multiplex PCR to produce amplicons enriched for target-site sequences; sequencing the amplicons and obtaining sample sequence data; subsequently executing on a processor, the steps of: receiving sample sequence data comprising a plurality of sequences; analyzing and merging of the sample sequence data and outputting merged sequences; developing target-site sequences containing predicted outcomes of repair events when a single-stranded or a double-stranded DNA oligonucleotide donor is provided and outputting the target predicted outcomes; binning the merged sequences with the target-site sequences or the optional target predicted outcomes using a mapper and outputting target-read alignments; re-aligning the binned target-read alignments to the target-site using an enzyme specific position-specific scoring matrix derived from biological data that is applied based on the position of a guide sequence and a canonical enzyme-specific cut site and producing a final alignment; analyzing the final alignment and identifying and quantifying mutations within a pre-defined sequence distance window from the canonical enzyme-specific cut sites; outputting the final alignment, analysis, and quantification results data as tables or graphics.

Many different arrangements of the various components and processes described herein as well as components or processes not shown, are possible without departing from the spirit and scope of the present disclosure. It should be understood that embodiments or aspects may include and otherwise be implemented by a combination of various hardware, software, or electronic components. For example, various microprocessors and application specific integrated circuits (“ASICs”) can be utilized, as can software of a variety of languages Also, servers and various computing devices can be used and can include one or more processing units, one or more computer-readable mediums, one or more input/output interfaces, and various connections (e.g., a system bus) connecting the components.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the methods and processes described herein include all actual or potential combinations of embodiments, aspects, options, examples, and preferences herein described. The methods described herein may omit any component or step, substitute any component or step disclosed herein, or include any component or step disclosed elsewhere herein. Should the meaning of any terms in any of the patents or publications incorporated by reference conflict with the meaning of the terms used in this disclosure, the meanings of the terms or phrases in this disclosure are controlling. Furthermore, the specification discloses and describes merely exemplary embodiments. All patents and publications cited herein are incorporated by reference herein for the specific teachings thereof.

### REFERENCES

1. Pinello, L. et al., “Analyzing CRISPR genome-editing experiments with CRISPResso.” Nat Biotechnol. 34(7): 695-697 (2016).
2. Lindsay, H. et al., “CrispRVariants: precisely charting the mutation spectrum in genome engineering experiments,” Nat. Biotechnol. 34(7): 701-703 (2015).
3. Labun, K. et al., “Accurate analysis of genuine CRISPR editing events with ampliCan Kornel,” bioRxiv 249474 (2018); now published in Genome Research 29:843-847 (2019)
4. Li, H., “Minimap2: Pairwise alignment for nucleotide sequences,” Bioinformatics 34(18): 3094-3100 (2018).
5. Shen, M. W. et al., “Predictable and precise template-free CRISPR editing of pathogenic variants,” Nature 563(7733): 646-651 (2018).
6. Hendel, A. et al., “Quantifying genome-editing outcomes at endogenous loci with SMRT sequencing.” Cell Rep. 7(1): 293-305 (2014).
7. Iyer, S. et al., “Precise therapeutic gene correction by a simple nuclease-induced double-stranded break,” Nature 568 (7753): 561-565 (2019).
8. Vu, G. T. H. et al., “Endogenous sequence patterns predispose the repair modes of CRISPR/Cas9-induced DNA double-stranded breaks in Arabidopsis thaliana,” Plant J. 92(1): 57-67 (2017).
9. Giannoukos, G. et al., “UDiTaS™, a genome editing detection method for indels and genome rearrangements,” BMC Genomics 19: 212 (2018).
10. Robinson, J., “Integrated genomics viewer,” Nat. Biotechnol. 29(1), 24-26 (2012).
11. Clement, K. et al., “Analysis and comparison of genome editing using CRISPResso2,” bioRxiv 1-20 (2018). Now published in Nat. Biotechnol. 37(3): 224-226 (2019)
12. Zetsche, B. et al., “Cpf1 Is a Single RNA-Guided Endonuclease of a Class 2 CRISPR-Cas System,” Cell 163(3): 759-771 (2015).
13. Liu, J. J. et al., “CasX enzymes comprise a distinct family of RNA-guided genome editors,” Nature 566(7743): 218-223 (2019).

### COMPUTER CODE

Exemplary code used to generate a 1D scoring matrix for gap open bonus during alignment using psnw.

```
def    self, cut_up, cut_down, rel_cut_pos, ref_amplicon, nuclease,
direction :
  ″″″
  This function takes in an minimum and maximum distance to look for CRISPR variants
(window size)
  and creates a string to introduce weights for gap open bonuses into the psnw
alignment algorithm.
  If there are multiple cuts, any position between the two cuts is weighted equally
  e.g. one cut Cas9
  :param cut_up: 2
  :param cut_down: 6
  :param rel_cut_pos: 4
  :param ref_amplicon: ACTGATCA
  :return: 002342100
  e.g. two cut Cas9
  :param cut_up: 2
  :param cut_down: 6
  :param rel_cut_pos: 4
  :param ref_amplicon: ACTGATCA
  :return: 001111100
  ″″″
  gap_open_string = [ ]
  cpfl_enz = list([″CAS12A″, ″CPF1″, ″MAD7″])
  try:
    # First the program looks to see if there is a second cut site
    if not None in rel_cut_pos:
      for i in range 0, len(ref_amplicon)):
        # If there is a second cut site, everything between the two cuts gets
a +1 bonus
        if cut_down <= i <= cut_up:
        gap_open_string.append(″1″)
        # If the position is not between the two cuts, it gets no bonus
        else:
          gap_open_string.append(″0″)
      # Execute these functions if there is only one cut site
      else:
      #If the enzyme is Cas9 nuclease, perform the following functions
      if ″CAS9″ in nuclease.upper:
        for i in range (0, len(ref_amplicon)):
          # The cut site gets a +4 gap open bonus automatically
          if i == int (rel_cut_pos[0]):
            gap_open_string.append(″4″)
          # Sites adjacent by 1bp to the cut site and distal from PAM get a
+3 gap open bonus
          elif ((direction[0 == ″forward″) and (i == (int(rel_cut_pos[0])−
1))) or \
              ((direction[0] == ″reverse″) and (i ==
(int(rel_cut_pos(0]) + 1))):
            gap_open_string.append(″3″)
          # Sites adjacent by 2bp to the cut site distal from PAM get a +2
gap open bonus
          elif ((direction[0] == ″forward″) and (i == (int(rel_cut_pos[0]) −
2))) or \
              ((direction(0) == ″reverse″ and (i ==
(int(rel_cut_pos[0] + 1))):
            gap_open_string.append(″2″)
          # Sites adjacent by 1bp to the cut site proximal from PAM get a +2
gap open bonus
          elif ((direction[0] == ″forward″) and (i == (int(rel_cut_pos[0]) +
1))) or \
              ((direction[0] == ″reverse″ and (i ==
(int(rel_cut_pos[0] - 1))):
            gap_open_string.append(″2″)
          # Sites between the upstream and downstream window get a +1 gap
open bonus
          elif cut_down <= i <= cut_up:
            gap_open_string.apend(″1″)
          # All other positions get no bonus
          else:
            gap_open_string.apend(″0″)
      elif any enzyme in nuclease.upper ( ) for enzyme in cpf1_enz):
        for i in range (0, len ref_amplicon)):
          if direction[0] == ″forward″:
            #Sites at the PAM distal cutsite get a +4 bonus
            if i == (int (rel_cut_pos[0]):
              gap_open_string.append(″4″)
            #Site at the PAM proximal cutsite get a +3 bonus
            elif i == (int.rel_cut_pos[0])− 4):
              gap_open_string.append (″3″)
            # Sites adjacent by 1bp to either cut site get a +2 gap open
bonus
            elif (i == (int(rel_cut_pos[0])−5)) \
                  or (i == (int(rel_cut_pos[0]−3))) \
                  or (i == (int(rel_cut_pos[0]) − 1)) \
                  or (i == (int(rel_cut_pos[0]) + 1 ):
                gap_open_string.append(″2″)
            # Sites between the upstream and downstream window get a +1
gap open bonus
            elif cut_down <= i <= cut_up:
              gap_open_string.append(″1″)
            # All other positions get no bonus
            else:
              gap_open_string.append(″0″)
            elif direction(0) == ″reverse″:
            # Sites at the PAM distal cutsite get a +4 bonus
            if i == (int(rel_cut_pos[0])):
              gap_open_string.append(″4″)
            # Site at the PAM proximal cutsite get a +3 bonus
            elif i == (int (rel_cut_pos[0]) + 4);
              gap_open_string.append(″3″)
            # Sites adjacent by 1bp to either cut site get a +2 gap open
bonus
            elif i == (int(rel_cut_pos[0]) + 5)) \
                  or (i == int(rel_cut_pos[0]) + 1)) \
                  or (i == int(rel_cut_pos[0]) − 1)) \
                  or (i == int(rel_cut_pos[0]) + 3)):
                gap_open_string.append(″2″)
            # Sites between the upstream and downstream window get a +1
gap open bonus
            elif cut_down <= i <= cut up:
              gap_open_string.append(″1″)
            # All other positions get no bonus
            else:
              gap_open_string.append(″0″)
      else:
        print (″Nuclease is not accepted: { }″).format(nuclease)
        raise ValueError
  except ValueError:
    print ″This value is not valid″:
  return ″″.join(gap_open_string)
```

Exemplary code used to generate 1D scoring matrix for gap extension bonus during alignment using psnw.

```
def get_gap_ext_bonus (self, cut_up, cut_down, rel_cut_pos, ref_amplicon):
  ″″″
  This function takes in an minimum and maximum distance to look for CRISPR variants
(window size)
  and creates a string to introduce weights for gap extension bonuses into the psnw
alignment algorithm.
  If there are multiple cuts, any position between the two cuts is weighted equally
  e.g. one cut Cas9
  :param cut_up: 2
  :param cut_down: 6
  :param rel_cut_pos: 4
  :param ref_amplicon: ACTGATCA
  :return: 001111100
  e.g. two cut Cas9
  :param cut_up: 2
  :param cut_down: 6
  :param rel_cut_pos: 4
  :param ref_amplicon: ACTGATCA
  :return: 001111100
  ″″″
  gap_ext_string = [ ]
  try:
    # First the program looks to see if there is a second cut site
    if not None in rel_cut_pos:
      for i in range (0, len(ref_amplicon)):
        # If there is a second cut site, everything between the two cuts gets
a +1 bonus
        if cut_down <= i <= cut_up:
          gap_ext_string.append(″1″)
        # If the position is not between the two cuts, it gets no bonus
        else:
          gap_ext_string.append(″0″)
    # Execute these functions if there is only one cut site
    else:
      for i in range (0, len(ref_amplicon)):
        # If there is a second cut site, everything between the two cuts gets
a +1 bonus
        if i == int(rel_cut_pos[0])):
          gap_ext_string.append(″1″)
        elif (i == (int(rel_cut_pos[0])−1)) or (i == (int(rel_cut_pos[0])+1)):
          gap_ext_string.append(″1″)
        elif cut_down <+ i <= cut_up:
          gap_ext_string.append(″1″)
        else:
          gap_ext_string.append(″0″)
  except ValueException :
    print (″This value is not valid″)
  return ″″.join(gap_ext_string)
```

What is claimed:

1. A computer implemented process for aligning biological sequences, the process comprising executing on a processor the steps of:
receiving sample sequence data comprising a plurality of sequences;
aligning the sequence data to a predicted target sequence using a matrix based on an enzyme specific position-specific scoring of a specific nuclease target sequence comprising a target site for one or more of Cas9, Cas12a, or other Cas enzymes, and wherein the matrix uses position-specific gap open and extension penalties;
outputting the alignment results as tables or graphics.

2. A computer implemented process according to claim 1, for identifying and characterizing double-stranded DNA break repair sites with improved accuracy, the process comprising executing on a processor the steps of:
(a) analyzing and merging of the sample sequence data and outputting merged sequences;
(b) developing target-site sequences containing predicted outcomes of repair events when a single-stranded or a double-stranded DNA oligonucleotide donor is provided and outputting the target-site sequences containing the predicted outcomes;
(c) binning the merged sequences with the target-site sequences containing the predicted outcomes using a mapper and outputting target-read alignments;
wherein the step of aligning the sequence data to a predicted target sequence using a matrix comprises re-aligning the target-read alignments to the target-site sequences containing the predicted outcomes using the matrix based on enzyme specific position-specific scoring of a specific nuclease target site, wherein the matrix is applied based on the position of a guide sequence and a canonical enzyme-specific cut site, and producing a final alignment;
the method further comprising:
(d) analyzing the final alignment and identifying and quantifying mutations within a pre-defined sequence distance window from the canonical enzyme-specific cut sites;
wherein the step of outputting the alignment results comprises outputting the final alignment, analysis, and quantification results data as tables or graphics, wherein the results show the percent editing, percent insertion, percent deletion, or a combination thereof.

3. The process of claim 1 or claim 2, wherein the sequence data comprises sequences from a population of cells or subjects.

4. The process of claim 2, wherein the pre-defined sequence distance window is enzyme specific and comprises between 1 nt to about 15 nt.

5. A method according to claim 2, further comprising the steps of:
extracting genomic DNA from a population of cells or tissue from a subject;
amplifying the genomic DNA using multiplex PCR to produce amplicons enriched for target-site sequences;
sequencing the amplicons and obtaining sample sequence data.

6. A system for selecting a guide RNA by identifying and characterizing double-stranded DNA (dsDNA) break repair sites with improved accuracy, the system comprising:
an electronic processor configured to:
(a) receive two or more guide RNAs;
(b) receive a genomic sample sequence data enriched for target-site sequences comprising a plurality of sequences, wherein the genomic sample sequence data enriched for target-site sequences is based at least in part on CRISPR-Cas editing, performed with the two or more guide RNAs, in a population of cells or tissue;
(c) merge the genomic sample sequence data enriched for target-site sequences;
(d) develop predicted target-site sequences for the genome containing predicted dsDNA break repair events based on a single-stranded or a double-stranded DNA oligonucleotide donor that is provided;
(e) bin, using a mapper, the merged sequences based on an alignment of each of the merged sequences to the genome and outputting binned target-read alignments;
(f) re-align the binned target-read alignments from step (e) to the predicted target-site sequences from step (d), wherein the binned target-read alignments are realigned using an aligner weighed by a Cas-enzyme-specific position-specific full gap open and gap extension multiple bonus scoring matrix, the aligner simultaneously and preferentially aligns multiple editing events within a defined sequence distance window of the predicted dsDNA break repair events for each Cas enzyme and each guide RNA and producing a final alignment;
(g) using the final alignment, identify and quantify editing events within the defined sequence distance window of the predicted dsDNA break repair events for each Cas enzyme and each guide RNA;
(h) select, from the two or more guide RNAs, one or more guide RNAs with effective CRISPR-Cas editing based on the quantification data from one or more selected from the group consisting of: the final alignment, percent editing, percent insertion, and percent deletion.

7. The system of claim 6, wherein the electronic processor is further configured to:
(i) use the one or more selected guide RNAs in further CRISPR-Cas editing experiments.

8. The system of claim 6, wherein the electronic processor is further configured to:
(j) output the final alignment, percent editing, percent insertion, percent deletion, or a combination thereof as tables or graphics.

9. The system of claim 6, wherein the multiple bonus scoring matrix uses position-specific gap open and gap extension variable penalty vectors to favor alignment of multiple editing events occurring at or near the predicted dsDNA break repair events.

10. The system of claim 6, wherein the multiple bonus scoring matrix is derived from biological editing data at canonical Cas enzyme cut sites and the position of each guide RNA.

11. The system of claim 1, wherein the genomic sample sequence data comprises sequences from a population of cells or subjects.

12. The system of claim 6, wherein the Cas enzyme is Cas9 or Cas12a.

13. The system of claim 6, wherein the sequence distance window of the predicted dsDNA break repair events for each Cas enzyme is between 1 nt to 15 nt.

14. The system of claim 12, wherein the defined sequence distance window of the predicted dsDNA break repair events for Cas9 is 8 nt.

15. The system of claim 12, wherein the defined sequence distance window of the predicted dsDNA break repair events for Cas12a is 9 nt from an offset center point-3 bp from a PAM distal cut.

16. The system of claim 6, wherein the Cas-enzyme specific position-specific matrix weights editing events that are closest to the predicted dsDNA break repair events with the lowest gap open or gap extension penalty according to a pre-defined matrix.

17. The system of claim 6, wherein the Cas-enzyme specific position-specific matrix has a gap open penalty of between 0 and +4.

18. The system of claim 1, wherein the Cas-enzyme specific position-specific matrix has a gap extension penalty of 0 or +1.

19. The system of claim 6, wherein the genomic sample sequence data is obtained by:
extracting the edited genomic DNA from the population of cells or tissue;
amplifying the edited genomic DNA using multiplex PCR to produce amplicons enriched for target-site sequences;
performing next generation sequencing of the amplicons enriched for target-site sequences and obtaining genomic sample sequence data enriched for target-site sequences.

20. A system for selecting a guide RNA by identifying and characterizing double-stranded DNA (dsDNA) break repair sites with improved accuracy, the system comprising:
an electronic processor configured to:
(a) receive a guide RNA;
(b) receive a genomic sample sequence data enriched for target-site sequences comprising a plurality of sequences, wherein the genomic sample sequence data enriched for target-site sequences is based at least in part on CRISPR-Cas editing, performed with the guide RNA, in a population of cells or tissue;
(c) merge the genomic sample sequence data enriched for target-site sequences;
(d) develop predicted target-site sequences for the genome containing predicted dsDNA break repair events based on a single-stranded or a double-stranded DNA oligonucleotide donor that is provided;
(e) bin, using a mapper, the merged sequences based on an alignment of each of the merged sequences to the genome and outputting binned target-read alignments;
(f) re-align the binned target-read alignments from step (e) to the predicted target-site sequences from step (d), wherein the binned target-read alignments are realigned using an aligner weighed by a Cas-enzyme-specific position-specific full gap open and gap extension multiple bonus scoring matrix, the aligner simultaneously and preferentially aligns multiple editing events within a defined sequence distance window of the predicted dsDNA break repair events for each Cas enzyme and the guide RNA and producing a final alignment;
(g) using the final alignment, identify and quantify editing events within the defined sequence distance window of the predicted dsDNA break repair events for each Cas enzyme and the guide RNA;
(h) determine whether the guide RNA exceeds a threshold, wherein the threshold is based on the quantification data from one or more selected from the group consisting of: the final alignment, percent editing, percent insertion, and percent deletion; and
(i) in response to the guide RNA exceeding the threshold, select, for use in further CRISPR-Cas editing experiments.
